(19) United States
(12) Patent Application Publication
(10) Pub. No.: US 2023/0138563 A1
(43) Pub. Date: May 4, 2023

(54) METHODS FOR QUANTIFICATION OF COMPOUNDS IN CULTIVARS OF Cannabis sp.

(71) Applicant: MEDLEAF BIOTECHNOLOGIES AND GLOBAL RESEARCH AND DEVELOPMENT INC., VICTORIA (CA)

(72) Inventors: DAVID NOSHAD, VICTORIA (CA); PAUL LI, VICTORIA (CA); ABDUL QUDEER, VICTORIA (CA)

(21) Appl. No.: 17/918,437

(22) PCT Filed: Apr. 14, 2021
(86) PCT No.: PCT/CA2021/050503

§ 371 Date: Oct. 12, 2022

Related U.S. Application Data
(60) Provisional application No. 63/009,590, filed on Apr. 14, 2020.

(51) Int. Cl. G01N 30/72; G01N 21/64; G01N 33/50; G01N 33/94

(52) U.S. Cl. CPC G01N 30/7206; G01N 21/6428; G01N 33/5058; G01N 33/948; G01N 33/5041; G01N 2030/884

(57) ABSTRACT

The disclosure provides a method for analyzing compounds extracted from Cannabis. The method comprises extracting cannabinoids or terpenes from a sample using a C₁-C₄ alcohol or C₅-C₈ solvent as an extraction solvent to produce a supernatant, drying the supernatant to produce a dried extract, and dissolving the dried extract in a second C₁-C₄ alcohol or C₅-C₈ solvent, separating the cannabinoids or terpenes by gas chromatography using a capillary column with hydrogen as a carrier gas; and detecting the cannabinoids or terpenes using a mass spectrometer. The disclosure also provides a method of determining an effect of one or more Cannabis-derived compounds on intracellular calcium concentration in a cell using a microfluidic device.

### FIELD

This disclosure relates to methods for identification and quantification of compounds derived from Cannabis. In particular, the disclosure relates to a gas chromatography method coupled with mass spectrometry for identification and quantification of compounds extracted from Cannabis, and a bioassay technique to determine the effects of Cannabis-derived compounds in a cell.

### BACKGROUND

Cannabis has been used for medical and recreational purposes for thousands of years. Medical Cannabis is legally available for patients in a number of countries (Lewis et al., 2017). The science of Cannabis is rapidly developing and recent evidence supports its therapeutic applications (Baron, 2018). A number of studies describe the biological potential of Cannabis for the treatment of pain, glaucoma, nausea, asthma, depression, insomnia and neuralgia (Duke and D Duke, 2002; Mechoulam et al., 1976), multiple sclerosis (Pryce and Baker, 2005), together with inflammatory diseases (Fichna et al., 2014; Costa et al., 2004), epilepsy (Devinsky et al., 2016), and movement disorders (Stampanoni et al., 2017).

Cannabis is a chemically rich plant of unparalleled versatility exhibiting a unique variety of natural compounds (Wang et al., 2017; Sohly, 2014). The compounds include cannabinoids (Appendino et al., 2011), terpenoids (Ross and Elsohly, 1996), flavonoids (Vanhoenacker et al., 2002), alkaloids (Turner and Elsohly, 1976), and others (Brenneisen, 2007). Cannabinoids, which are a group of compounds bearing a C₂₁ terpenophenolic skeleton, generate the medically important chemicals of the Cannabis plant. One example of these compounds is cannabidiol (CBD), which may have efficacy in several pathologies, such as, for example, inflammatory and neurodegenerative diseases, epilepsy, autoimmune disorders such as multiple sclerosis, arthritis, schizophrenia and cancer (Pisanti et al., 2017). Migration and aggressiveness of cell propagation, migration, invasion and anomalous cell death in these pathologies may be associated with oscillations in intracellular calcium storages (Montana and Sontheimer, 2011; Watkins and Sontheimer, 2012), which can be affected by Cannabis-derived compounds. For example, intracellular Ca²⁺ accumulation is viewed as a vital element in the development of neurodegenerative diseases (Duncan et al., 2010). However, current methods for measuring intracellular Ca²⁺ are often time-consuming. Thus, there remains a need for a bioassay to determine the concentration of intracellular calcium in a cell, as a function cannabinoid or terpene added to the cell, as an analytical tool for testing the effectiveness of Cannabis compounds in the treatment of various diseases and disorders.

Apart from cannabinoids, a number of terpenes found in Cannabis have also been reported to act synergistically with cannabinoids in the treatment of pain, inflammation, depression, anxiety, addiction, epilepsy, cancers, and infections (Russo, 2011). Around 200 terpenes have been reported in Cannabis (Ross and Elsohly, 1996; Ibrahim et al., 2019). In foods and Cannabis-filled foods, terpenes are mainly used as flavours, but most of them are lost due to food processing and thus, addition of these compounds after processing is a common practice (King, 2019). In addition, terpenes show synergistic effects with cannabinoids. For example, limonene, pinene, caryophyllene, and myrcene combined with CBD may be used as an antiseptic for social anxiety disorder and acne therapies (Aizpurua-Olaizola et al., 2016). Moreover, terpenes demonstrate anti-cancer, anti-fungal, anti-viral, anti-inflammatory, and anti-parasitic properties (Gallily et al., 2018; Casano et al., 2011). Due to the volatile nature of terpenes, gas chromatography (GC) is often used for their determination. Terpenes have been mostly identified by gas chromatography mass spectroscopy (GC-MS) and headspace-solid phase microextraction (HS-SPME coupled with GC-MS) (Ibrahim et al., 2018; Arnoldi et al., 2017; Booth et al., 2017; Calvi et al., 2018), GC-flame ionization detection (GC-FID) (Richins et al., 2018), or direct injection of hemp oil extract into GC-MS (Pavlovic et al., 2018).

However, there remains a need for detecting and quantifying the presence and amount of various compounds, including cannabinoids and terpenes, in a single sample.

### SUMMARY

Various aspects of the present disclosure provide a method for analyzing a sample containing cannabinoids, the method comprising: extracting the cannabinoids from the sample using a first C₁-C₄ alcohol as an extraction solvent to produce a supernatant, drying the supernatant to produce a dried extract, and dissolving the dried extract in a second C₁-C₄ alcohol; separating the cannabinoids by gas chromatography using a capillary column with hydrogen as a carrier gas; and detecting the cannabinoids using a mass spectrometer.

Various aspects of the present disclosure further comprise quantifying the amount of CBD, CBC, CBG, CBN and/or THC in the sample using CBD-d3, CBC-d3, CBG-d3, CBN-d3 and/or THC-d3, respectively, as internal standards. Additional aspects of the present disclosure further comprise quantifying the amount of CBC, CBG and/or CBN in the sample using a standard addition method.

Various aspects of the present disclosure provide a method of detecting more than one cannabinoid in a sample, the method comprising: extracting the more than one cannabinoid from the sample using a first C₁-C₄ alcohol as an extraction solvent to produce a supernatant, drying the supernatant to produce a dried extract, and dissolving the dried extract in a second C₁-C₄ alcohol; separating the more than one cannabinoid by gas chromatography using a capillary column with hydrogen as a carrier gas; and detecting the more than one cannabinoid using a mass spectrometer.

In various embodiments, a flow rate of the carrier gas is constant at about 1.6 mL/minute.

In various embodiments, a temperature program of the column is an initial temperature of 180° C. for 0.5 minutes, a first ramp of 5° C./minute to 250° C., and a second ramp of 10° C./minute to a final temperature of 325° C. for 2 minutes.

In various embodiments, the method further comprises quantifying the amount of CBD and/or THC in the sample using CBD-d3 and/or THC-d3, respectively, as internal standards.

In various embodiments, the method further comprises quantifying the amount of CBC, CBG and/or CBN in the sample using a standard addition method.

In various embodiments, an injection volume for the column is about 1 μL.

In various embodiments, the first and second C₁-C₄ alcohols are methanol.

In various embodiments, a split ratio of an injector of the column is about 5:1.

In various embodiments, a temperature of an injector of the column is about 280° C.

In various embodiments, detector port temperatures of the mass spectrometer are about 280° C. at a transfer line, about 230° C. at an ion source, and about 150° C. at a quadrupole.

In various embodiments, the sample contains five or more cannabinoids and five of the cannabinoids are identified in the sample.

In various embodiments, the extraction step comprises suspending the sample in the C₁-C₄ alcohol, vortexing, sonicating and centrifuging the sample to produce the supernatant and filtering the supernatant.

Various aspects of the present disclosure provide a method of detecting more than one terpene in a sample, the method comprising: extracting the more than one terpene from the sample using a first C₅-C₈ solvent as an extraction solvent to produce a supernatant, drying the supernatant to produce a dried extract, and dissolving the dried extract in a second C₅-C₈ solvent; separating the more than one terpene by gas chromatography using a capillary column with hydrogen as a carrier gas; and detecting the more than one terpene using a mass spectrometer.

In various embodiments, a flow rate of the carrier gas is constant at about 1.6 mL/minute.

In various embodiments, a temperature program of the column is an initial temperature of 70° C., a first ramp of 10° C./minute to 90° C., a second ramp of 40° C./minute to 150° C., and a third ramp of 120° C./minute to a final temperature of 300° C.

In various embodiments, an injection volume for the capillary column is about 1 μL.

In various embodiments, the first and second C₅-C₈ solvents are hexane.

In various embodiments, a split ratio of an injector of the column is about 5:1.

In various embodiments, detector port temperatures of the mass spectrometer are about 280° C. at a transfer line, about 230° C. at an ion source, and about 150° C. at a quadrupole.

In various embodiments, the extraction step comprises suspending the sample in the C₅-C₈ solvent, vortexing, sonicating and centrifuging the sample to produce the supernatant and filtering the supernatant.

In various embodiments, the sample contains seven or more terpenes and seven of the terpenes are identified in the sample.

In various embodiments, the dimensions of the capillary column are 30 m×0.25 mm×0.25 μm.

In various embodiments, a stationary phase of the capillary column is (5%-phenyl)-methylpolysiloxane.

In various embodiments, the sample is dried flowers of a Cannabis plant.

In various embodiments, the mass spectrometer is a quadrupole mass spectrometer.

Various aspects of the present disclosure provide a method of determining an effect of one or more Cannabis-derived compounds on intracellular calcium concentration in a cell, the method comprising: isolating a cell in a microfluidic device; measuring fluorescence of the cell to determine a background fluorescence (Fₘᵢₙ); adding a cell-permeable fluorescent calcium indicator to a reservoir in the microfluidic device; measuring fluorescence of the cell and determining a first intracellular calcium concentration in the cell according to equation (1):

$$[ {Ca}^{2 +} ] = {K}_{d} ( \frac{F - {F}_{m i n}}{{F}_{m a x} - F} ) , \qquad ( 1 )$$

adding the one or more Cannabis-derived compounds to the reservoir in the microfluidic device; measuring fluorescence of the cell and determining a second intracellular calcium concentration in the cell according to equation (1); adding ionomycin to the cell; measuring fluorescence of the cell to determine a maximum fluorescence (Fₘₐₓ); and comparing the first intracellular calcium concentration to the second intracellular calcium concentration to determine the effect of the one or more Cannabis-derived compounds on intracellular calcium concentration in the cell.

In various embodiments, the one or more Cannabis-derived compounds are cannabinoids and/or terpenes. For example, the Cannabis-derived compound is CBD. For example, the one or more Cannabis-derived compounds are CBD and myrcene.

In various embodiments, the cell-permeable fluorescent calcium indicator is Fluo-4 acetoxymethyl ester (Fluo-4 AM).

In various embodiments, the cell is a glioma cell.

Other aspects and features of the present invention will become apparent to those of ordinary skill in the art upon review of the following description of specific embodiments of the invention in conjunction with the accompanying claims.

### BRIEF DESCRIPTION OF THE DRAWINGS

In drawings which illustrate embodiments of the disclosure,

FIG. 1 shows the chemical structure of various compounds in Cannabis: (A) CBL, (B) CBD, (C) CBC, (D) THC, (E) CBG, and (F) CBN.

FIG. 2 shows the chemical structure of various compounds in Cannabis: (A) α-pinene, (B) β-pinene, (C) myrcene, (D) limonene, (E) β-caryophyllene and (F) humulene.

FIG. 3 shows a mass spectrum of cannabidiol (CBD).

FIG. 4 shows a mass spectrum of tetrahydrocannabinol (THC).

FIG. 5 shows a GC-MS chromatogram of cannabinoid standards. SIM data (ion m/z in parentheses) was obtained for CBD (231), CBC (231), CBG (193) and CBN (295). The x-axis represents time in minutes and the y-axis represents ion abundance.

FIG. 6 shows a further GC-MS chromatogram of a cannabinoid standard. SIM data (ion m/z in parentheses) was obtained for CBD (231), CBC (231) and THC (299). The x-axis represents time in minutes and the y-axis represents ion abundance.

FIG. 7 shows four gas chromatograms of isotope standards: (a) CBD-d3 at m/z 231 and 234; and (b) THC-d3 at m/z 193 and 196.

FIG. 8 shows a GC-MS chromatogram of terpenes. The x-axis represents time in minutes and the y-axis represents ion abundance.

FIG. 9 shows a schematic of a microfluidic single-cell device showing reservoirs (1, 2, 3), channels (4, 5, 6) and cell chamber (7) according to an embodiment of the invention.

FIG. 10 shows cell calcium changes in a glioma cell stimulated by two different concentrations of CBD. The cell was first loaded with Fluo-4 AM ester (5 μM), then treated with CBD (9.5 and 19 μM), followed by ionomycin (10 μM). The cell was stained to blue when treated with trypan blue.

FIG. 11 shows cell calcium changes in a glioma cell stimulated by myrcene (20 μM), CBD (20 μM) and both myrcene and CBD together (20 μM), followed by ionomycin (10 μg/mL) in 50 μM CaCl₂.

### DETAILED DESCRIPTION

In the context of the present disclosure, various terms are used in accordance with what is understood to be the ordinary meaning of those terms.

Disclosed embodiments include systems, apparatus and methods for identification and quantification of compounds extracted from Cannabis. For example, the compounds may be cannabinoids or the compounds may be terpenes. Various embodiments as disclosed herein are directed to a fast and efficient gas chromatography (GC) method coupled with mass spectrometry (MS) for identification and quantification of compounds extracted from Cannabis, such as, for example, cannabinoids and terpenes. The embodiments as disclosed herein allow simultaneous separation, identification and quantification of major cannabinoids such as, for example, cannabidiol, (CBD), cannabichromene (CBC), tetrahydrocannabinol (THC), cannabigerol (CBG), and cannabinol (CBN) by means of GC-MS. For example, the methods as described herein achieve better separation between CBD and CBC than prior art methods, and allow for the simultaneous identification of, for example, five different cannabinoids, as compared to previous methods. In various embodiments, the methods disclosed herein are suitable for chemical profiling of cannabinoids extracted from different types of Cannabis plant materials. For example, the methods as disclosed herein may be used for chemical profiling of cannabinoids from dried flowers of different Cannabis varieties. The embodiments as disclosed herein may allow for simultaneous separation, identification and/or quantification of terpenes such as, for example, α-pinene, β-pinene, myrcene, limonene, 4-chlorophenol, β-caryophyllene and humulene by means of GC-MS. In various embodiments, the methods disclosed herein are suitable for chemical profiling of terpenes extracted from different types of Cannabis plant materials. For example, the methods as disclosed herein may be used for chemical profiling of terpenes from dried flowers of different Cannabis varieties. Disclosed embodiments also include a bioassay to determine the concentration or potency of Cannabis compounds in a cell and the effect of these compounds on intracellular calcium concentration.

In various embodiments, the GC coupled with MS methods as disclosed herein allow simultaneous detection of cannabinoids and/or terpenes in a variety of samples. The methods may be fast and efficient for simultaneous detection of THC, as well as non-tetrahydrocannabinoids, such as, for example, CBD, CBC, CBG and CBN in complex plant matrices. The methods may be fast and efficient for simultaneous detection of α-pinene, β-pinene, myrcene, limonene, 4-chlorophenol, β-caryophyllene and humulene in complex plant matrices.

In various embodiments, the methods comprise extracting the cannabinoids from the sample using methanol as an extraction solvent to produce a supernatant, drying the supernatant to produce a dried extract, and dissolving the dried extract in methanol; separating the cannabinoids by gas chromatography using a capillary column with hydrogen as a carrier gas; and detecting the cannabinoids using a quadrupole mass spectrometer.

In various embodiments, the methods comprise extracting the terpenes from a sample using hexane as an extraction solvent to produce a supernatant, drying the supernatant to produce a dried extract, and dissolving the dried extract in hexane; separating the terpenes by gas chromatography using a capillary column with hydrogen as a carrier gas, and detecting the terpenes using a quadrupole mass spectrometer.

Also provided herein is a bioassay to determine intracellular calcium concentration in a cell. Cannabinoids and terpenes are important classes of Cannabis-derived compounds that have a diverse range of pharmacological properties. The pharmacological properties of cannabinoids, such as cannabidiol, and terpenes, may be measured using a single-cell microfluidic approach. Various concentrations of cannabinoid and/or terpene may be evaluated to excite an increase in intracellular calcium levels in various cell lines, such as in the human glioma cell line U87 MG. lonomycin may be used as a control to saturate intracellular calcium required for calibration of the concentration. In various embodiments, real-time measurement results suggested that CBD produces an increase in intracellular calcium concentration signal in real time, signifying the single-cell microfluidic bioassay may be used to investigate pharmacological properties of various Cannabis-derived compounds.

### EXAMPLES

These examples illustrate various aspects of the invention, evidencing a variety of conditions for chemical profiling of different Cannabis varieties, and for separately identifying and quantifying different compounds such as, for example, cannabinoids and/or terpenes. The examples also demonstrate a bioassay technique for measuring intracellular calcium in a cell in real-time, and the effect of Cannabis-derived compounds on this concentration. Selected examples are illustrative of advantages that may be obtained compared to alternative methods, and these advantages are accordingly illustrative of particular embodiments and not necessarily indicative of the characteristics of all aspects of the invention.

As used herein, the term “about” refers to an approximately ±10% variation from a given value. It is to be understood that such a variation is always included in any given value provided herein, whether or not it is specifically referred to.

### Example 1

### Methods of Detection of Cannabinoids and Terpenes in a Cannabis Sample

Standards and Reagents

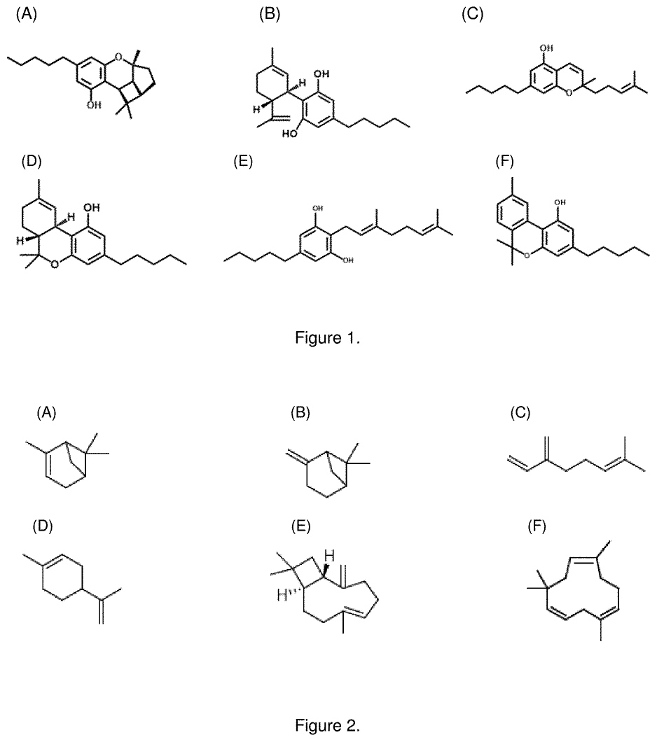

Cannabinoid standards (CBL, CBD, CBC, THC, CBG, CBN, CBD-d3 and THC-d3) were purchased from Cerilliant Corporation (Round Rock, Tex.) as drug enforcement agency-exempt solutions, i.e. 1 mg/mL solution in methanol (MeOH). The structures of the cannabinoids are shown in FIG. 1. Terpene standards (α-pinene, β-pinene, myrcene, limonene, 4-chlorophenol, β-caryophyllene and humulene) were purchased from Sigma-Aldrich. The structures of the terpenes are shown in FIG. 2. All other chemicals and solvents used were of analytical grade.

Gas Chromatography (GC-MS) Analysis

The cannabinoids and terpenes were determined using GC-MS. The GC (Agilent 6890 series) was equipped with a HP-5MS column (30 m×0.25 mm, 0.25 μm film thickness). Hydrogen was used as the carrier gas at a constant flow of 1.6 mL/min. The oven temperature for cannabinoid detection was programmed from 180° C. (for 0.50 min) to 250° C. at 5° C./min, and then to a final temperature of 325° C. (at 10° C./min) which was maintained for 2 min. One μL of sample was injected using an autosampler and the injector port temperature was set to 280° C. The oven temperature for terpene detection was programmed from 70° C. to 90° C. (at 10° C./min), then to 150° C. (at 40° C./min) and to 300° C. (at 120° C./min). Detector parameters were MS source at 230° C. and MS Quad at 150° C.

The MS (model 5973N) used electron impact ionization and transmission quadrupole mass spectrometer. For quantification, data was obtained using the selected ion monitoring (SIM) method.

Cannabis Plant Extraction

For cannabinoid detection, dried flowers of different cannabis varieties were ground using a mortar and pestle, and samples of 200 mg were accurately weighed. For extraction, samples were suspended in 2 ml methanol, followed by vortexing, sonication for 10-20 min, and centrifugation at 4,000 rpm for 5 min; the supernatants were transferred to a 10 mL glass vial. The entire procedure was repeated two more times and the respective supernatants were combined. Thereafter, supernatants were filtered by passing through a 0.22-μm sterile syringe filter, and dried under a gentle stream of nitrogen gas. Dried weights of various samples after extraction were obtained: C. sativa (92.2 mg), C. indica (35.1 mg), Cannalope Kush (72.5 mg), Cannabis 5-CW (Charlotte Web) (56.3 mg), Rock Star (74.2 mg) and Super Silver (66.8 mg). Dried extracts were reconstituted with 200 μl MeOH for GC-MS analysis.

For terpene detection, dried flowers of different cannabis varieties were ground using a mortar and pestle, and samples of 100 mg accurately weighed. For extraction, samples were suspended in 2 mL hexane, followed by vortexing, and sonication for 10-20 min, and centrifugation at 4,000 rpm for 5 min. The supernatants were then transferred to a 10 mL glass vial. Dried extracts were reconstituted with hexane for analysis by GC-MS.

Standard Solutions of Cannabinoids

Stock solutions of individual standards and internal standards were prepared separately at concentration of 100 μg/mL in methanol. A standard mixture of the cannabinoid standard and internal standard (100 μg/mL) were also prepared.

Spiking of Cannabis Extracts with Cannabinoid Standards

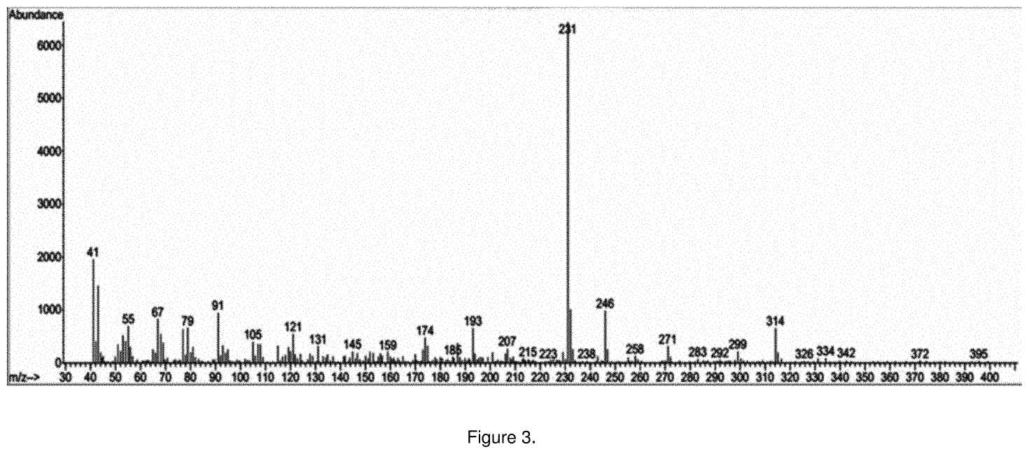

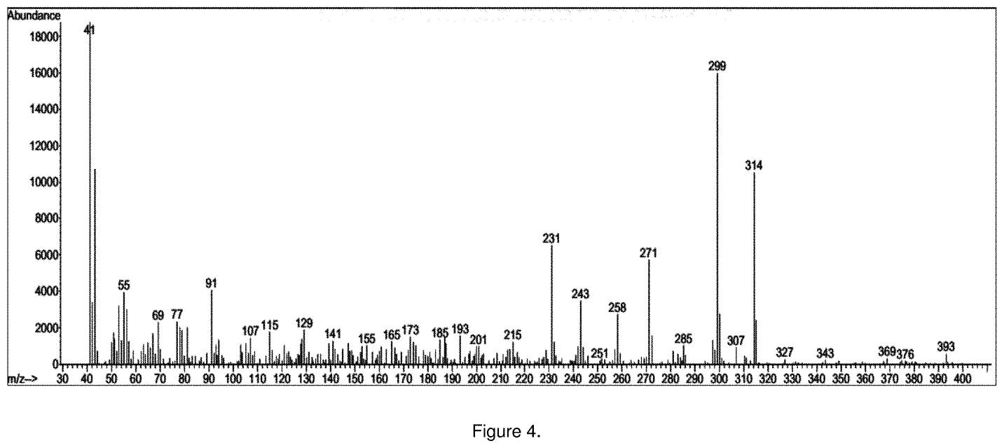

Cannabinoids were quantified using internal standard and standard addition methods. For standard addition, C. indica, C. sativa, Cannalope Kush, Cannabis 5-CW, Rock Star and Super Silver extracts (14 mg/ml) were added (or spiked) with cannabinoid standards i.e. CBC, CBG, and CBN (50 μg/ml and 100 μg/ml), using CBD-d3 (50 μg/ml) as the internal standard. For THC quantification, C. indica, C. sativa, Cannalope Kush, Cannabis 5-CW, Rock Star, Super Silver extracts (14 mg/ml) were quantified using THC-d3 (50 μg/ml) as the internal standard. For CBD quantification, C. indica, C. sativa, Cannalope Kush, Rock Star, Super Silver extracts (14 mg/ml) and Cannabis 5-CW extract (0.2815 mg/ml) were quantified using CBD-d3 (50 μg/ml) as the internal standard. CBL was below detection level and it was not quantified. Data was obtained using selected ion monitoring (SIM), and quantified using ions in m/z in parentheses for CBL (231), CBD (231), CBC (231), THC (193), CBG (193), and CBN (295). FIGS. 3 and 4 show the mass spectra obtained for CBD and THC, respectively.

Results for Cannabinoid Detection

The disclosure provides analytical methods for chemical profiling of different Cannabis varieties, and for separating and identifying different cannabinoids. Cannabinoids were identified by comparing mass spectra with an online compound database search and retention times of cannabinoids with their corresponding standard compounds. The disclosed methods provide faster analysis and better separation of cannabinoids. For example, the disclosed methods provided faster separations of CBD, CBC, THC, CBG, and CBC (within 13 min) than the methods reported by Richins et al., 2018 (24 min), Leghissa et al., 2017 (18 min), Mariotti et al., 2016 (20.5 min), Cadola et al., 2013 (19 min), and Hillig et al., 2004 (26.5 min). Moreover, CBD and CBC were baseline separated (resolution of 1.5), which was better than in methods published by Mariotti et al., 2016, Hillig et al., 2004, and Ilias et al., 2004 (resolution less than 1).

Thus, the methods disclosed herein provide a fast GC-MS methodology which ensures high separation efficiency (or resolution) and allows for the simultaneous quantification of compounds from complex Cannabis plant matrices. For example, the methods as disclosed herein may provide for quantification of five compounds from Cannabis plant matrices.

Identification of Cannabinoids by GC-MS

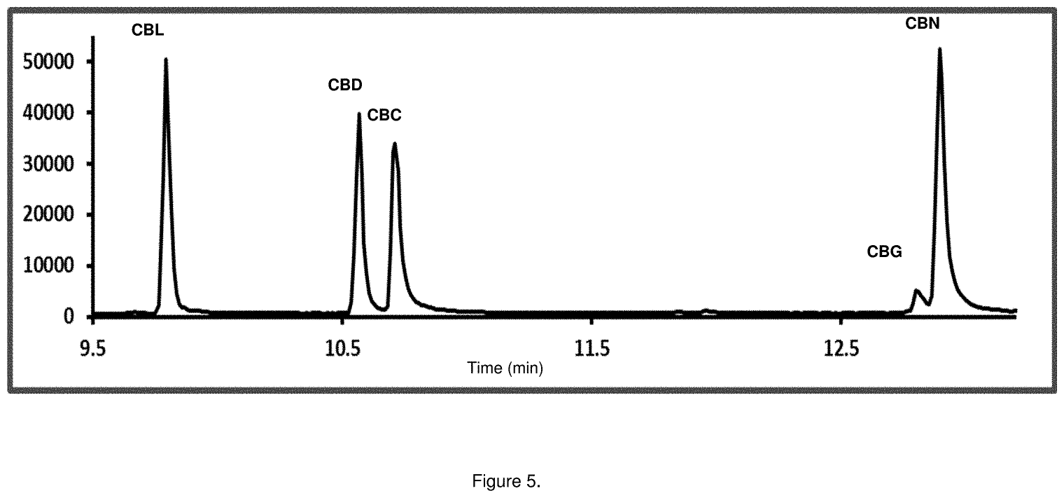

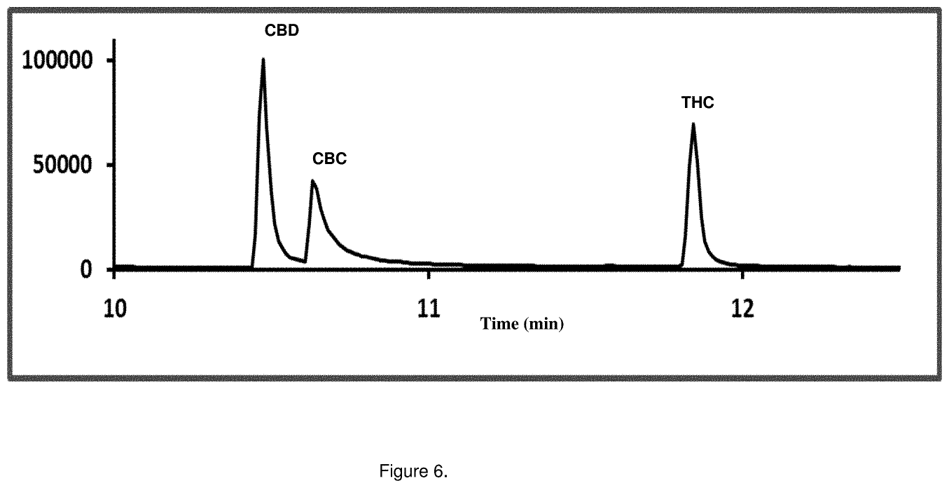

Gas chromatography-mass spectrometry (GC-MS) was used for identification and quantification of cannabinoids. FIGS. 5 and 6 show GC-MS chromatograms of cannabinoid standards. The identified compounds were CBL, CBD, CBC, THC, CBG and CBN. Different samples such as C. sativa, C. indica, Cannalope Kush, Cannabis 5-CW, Super Silver and Rock Star were analyzed for identification of target cannabinoids. The criteria for identification of the target constituents were retention time in correspondence to standards and mass spectral data library search. CBD, CBC, THC, CBG, and CBN were identified from tested Cannabis samples. Target compounds were identified in the elution order of (i) CBD (C₂₁H₃₀O₂; molecular mass 314.469 g/mol), (ii) CBC (C₂₁H₃₀O₂; molecular mass 314.469 g/mol), (iii) THC (C₂₁H₃₀O₂; molecular mass 314.469 g/mol), (iv) CBG (C₂₁H₃₂O₂; molecular mass 316.485 g/mol), and (v) CBN (C₂₁H₂₆O₂; molecular mass 310.4319 g/mol). CBD is abundant and is the major compound of Cannabis 5-CW; whereas THC is the major compound in the rest of the samples. Five different cannabinoids (CBD, CBC, THC, CBG, and CBN) may be analyzed at once in the methods disclosed herein, but other studies have focused on different cannabinoids using other techniques such as GC-FID. For instance, the recent work by Pellati et al., 2018 analyzed cannabinoids and terpenes but they excluded THC, CBC and CBN. Similarly, Bruci et al. (2012), Vanhove et al. (2011), and Tipparat et al. (2012, 2014), only analyzed CBD, THC, CBN, but not CBC and CBG.

Quantification of Cannabinoids from Cannabis Samples

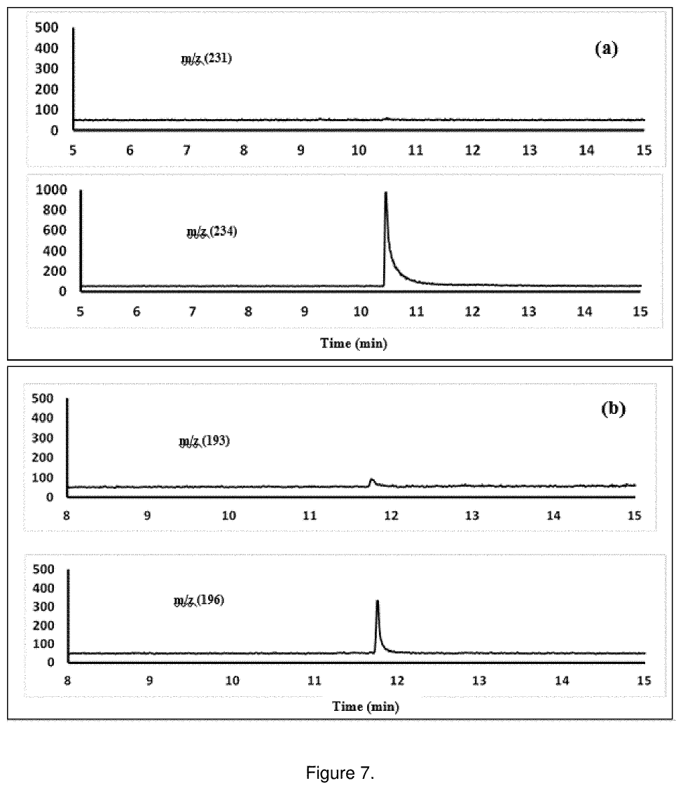

The methods described herein were successfully applied for quantification of cannabinoids from different Cannabis samples. Phytocannabinoids were quantified by standard addition and internal standard methods. THC was quantified using THC-d3 as the internal standard; whereas CBD, CBL, CBC, CBG and CBN were quantified using the CBD-d3 internal standard. Both THC-d3 and CBD-d3 were found to be reasonably pure because THC-d3 contains the 196 peak but a little 193 peak, and CBD-d3 contains the 234 peak but no 231 peak, see FIG. 7. CBL was below the detection limit (<0.02% dry weight). FIG. 7 shows gas chromatograms of isotope standards CBD-d3 at m/z 231 and 234, and THC-d3 at m/z 193 and 196.

Table 1 showed quantification data of cannabinoids from different Cannabis samples. As listed in Table 1, Cannabis 5-CW showed the highest CBD content (16.43%) and Super Silver the lowest (0.08%). The THC content ranged from 3.5% in Cannalope Kush and Rock Star to 2.71% in Cannabis 5-CW. The CBC levels were the highest in Cannabis 5-CW (4.15%) and the lowest in C. indica (0.39%). Interestingly, CBG was recorded the highest level for Cannalope Kush (4.18%) and the lowest for Cannabis 5-CW (0.39%). The highest concentration of CBN was seen in the case of Rock Star (1.29%) and the lowest level was found in Cannabis 5-CW (0.37%). This becomes evident from Table 1 that Super Silver and Rock Star are rich in CBG and CBN, respectively, and so these two samples should be further tested for their pharmacological effects. Though other samples have high contents of CBD or THC, none of those samples showed high concentrations of CBG and CBN.

TABLE 1
Cannabinoids concentrations (% dry weight) determined in Cannabis samples by GC-MS.

| | GC-MS estimated cannabinoids (Mean ± SD) | | | | | |
|---|---|---|---|---|---|---|
| Samples | % CBD | % THC | % CBC | % CBG | % CBN | % CBL |
| Cannabis sativa | 0.100 ± 0.004 | 3.45 ± 0.18 | 0.92 ± 0.09 | 0.72 ± 0.17 | 0.88 ± 0.44 | <0.02 |
| Cannabis indica | 0.09 ± 0.05 | 3.20 ± 0.10 | 0.39 ± 0.03 | 0.84 ± 0.22 | 0.38 ± 0.13 | <0.02 |
| Cannalope Kush | 0.10 ± 0.01 | 3.50 ± 0.04 | 1.09 ± 0.21 | 4.18 ± 1.25 | 0.54 ± 0.10 | <0.02 |
| Cannabis 5-CW | 16.43 ± 0.82 | 2.71 ± 0.12 | 4.15 ± 0.02 | 0.39 ± 0.13 | 0.37 ± 0.16 | <0.02 |
| Rock Star | 0.100 ± 0.003 | 3.50 ± 0.05 | 0.45 ± 0.02 | 0.59 ± 0.05 | 1.29 ± 0.38 | <0.02 |
| Super Silver | 0.08 ± 0.01 | 3.40 ± 0.20 | 0.69 ± 0.17 | 2.25 ± 0.70 | 0.67 ± 0.05 | <0.02 |

C. sativa contains cannabinoids of CBD (0.10%), THC (3.45%), CBC (0.92%), CBG (0.72%), and CBN (0.88%). C. indica the cannabinoids levels were CBD (0.09%), THC (3.20%), CBC (0.39%), CBG (0.84%), and CBN (0.38%). Cannalope Kush cannabinoids levels were CBD (0.10%), THC (3.50%), CBC (1.09%), CBG (4.18%), and CBN (0.54%). Phytocannabinoids in 5-CW were in the range of CBD (16.43%), THC (2.71%), CBC (4.15%), CBG (0.39%), and CBN (0.37%). In case of Rock Star cannabinoids were CBD (0.10%), THC (3.50%), CBC (0.45%), CBG (0.59%), and CBN (1.29%). The levels of cannabinoids in Super Silver cannabinoids were CBD (0.08%), THC (3.40%), CBC (0.69%), CBG (2.25%), and CBN (0.67%), respectively. Different Cannabis cultivars showed cannabinoids contents in range of CBD (9.84-0.01%), THC (21.53-0.26%), low CBC (0.62-0.03%), CBG (2.08-0.05%) (Richins et al., 2018), and CBN (7.25-0.18%) (Wang et al., 2017). Chemical composition of Cannabis varieties depends upon several factors such as genetic structure, soil, climate, maturity of plants at harvest and conditions at which plants were stored. Seasonal variations affect the levels of CBN and THC in Indiana varieties of Cannabis (Phillips et al., 1970). Moreover, plant age, time of collection and geographic location are also among the factors affecting chemical composition of cannabis (Holley et al., 1975).

The above examples demonstrate that the disclosed GC-MS methods provide for chemical profiling of cannabinoids from a variety of Cannabis samples. The disclosed methods may be used for both identification and quantification of cannabinoids. Among the samples tested above, CBD and THC were predominant constituents. Cannabis 5-CW exhibited the highest CBD level. On the other hand, Cannalope Kush and Rock Star showed the highest THC levels as compared to other Cannabis samples. Moreover, the methods as disclosed herein may also provide phytochemical characteristics of Cannabis plants.

Results for Terpene Detection

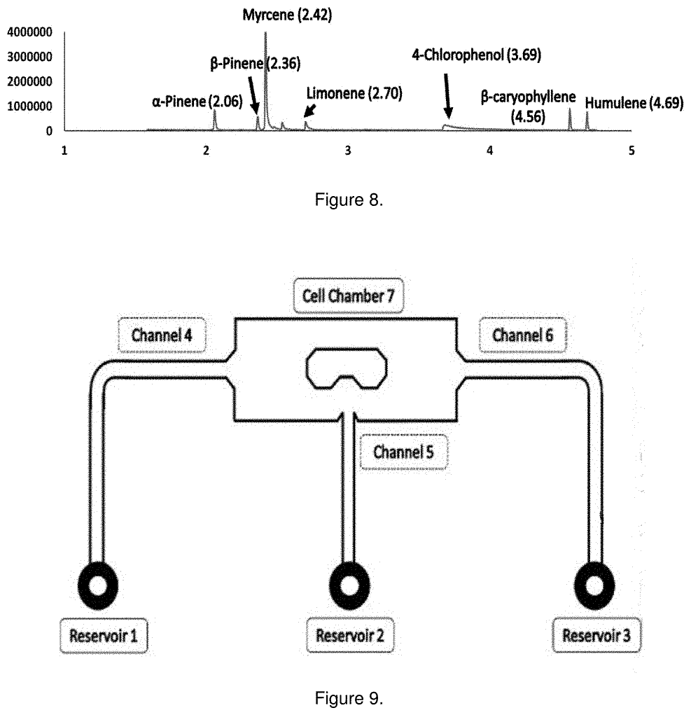

A standard mixture of terpenes was analyzed by GC-MS. As shown in FIG. 8, the method of separation of terpenes by GC-MS using a temperature program of the column comprising an initial temperature of 70° C., a first ramp of 10° C./minute from 70° C. to 90° C., a second ramp of 40° C./minute from 90° C. to 150° C. and a third ramp of 120° C./minute from 150° C. to 300° C. allowed for the simultaneous identification of compounds within a short time and with high separation resolution. This method provides for a faster analysis and better separation of terpenes compared to other prior art methods, and in particular, for the analysis of α-pinene, β-pinene, myrcene, limonene, 4-chlorophenol, β-caryophyllene and humulene. Separation of these compounds according to the methods disclosed herein may be achieved, for example, within 5 minutes. For example, separation as shown in FIG. 8 was achieved in 4.75 minutes. This is faster than previous methods reported by Arnoldi et al, 2017 (separation achieved in 8 minutes), Richins et al., 2018 (separation achieved in 14 minutes), Booth et al., 2017 (separation achieved in 8 minutes), Ibrahim et al., 2018 (separation achieved in 40 minutes). Additionally, β-pinene and myrcene were baseline-separated, with a retention time difference of 0.06 minutes and a resolution of 1.5. This level of separation is improved compared to the method of Honnold et al., 2017 which had a retention time difference between these two compounds of 0.028 minutes and peak resolution was unclear as the compounds were not baseline separated and had overlapping effects. The methods disclosed herein use a moderate initial temperature of 70-90° C. and a higher ramping temperature (40° C. per minute and 120° C./minute as compared to other methods which use a lower initial temperature (45° C. to 50° C.) or higher initial temperature (200° C.) with a slower ramping temperature (approximately 20° C. maximum).

### Example 2

### Bioassay to Determine Concentration of CBD and Terpenes in a Cell

The current study was designed to investigate the pharmacological potential of CBD on calcium uptake in U87MG glioma cells by a method using a single-cell microfluidic approach.

Chip Fabrication and Characterization

The glass chip was fabricated through the standard micromachining processes at Canadian Microelectronic Corporation (CMC) by a process that includes standard chip cleaning, thin film deposition, photolithography, photoresist development, hydrofluoric acid wet etching, reservoir forming, and chip bonding, as previously reported (Li et al. 2005). The chip design is shown in FIG. 9, consisting of three reservoirs, three channels and one chamber containing the cell retention structure to isolate the single cell. Reservoir 1 was used for cell introduction and washing, reservoir 2 was used for reagent delivery, and reservoir 3 was a waste reservoir. The channel was 40 μm deep, while the reservoirs were 600 μm deep and 2.5 mm in diameter.

Reagents and Cell Samples

A fluorescent calcium probe, Fluo-4 AM ester (50 μg, special packaging, Molecular Probes, Eugene, Oreg.) was first dissolved in 50 μL of dimethyl sulfoxide (DMSO, 99.9%, Sigma-Aldrich, St. Louis, Mo.) to make a stock solution of 1 μg/μL. Before use, it was freshly diluted in Hanks' balanced salt solution (HBSS, Invitrogen Corp., Grand Island, N.Y.) to make a 5.0 μM working solution. Due to light sensitivity of Fluo-4 AM, it must be stored in the dark at −20° C. Cannabidiol (CBD) was purchased from Cerilliant Corporation (Round Rock, Tex.) as drug enforcement agency-exempt solution, i.e. 1 mg/mL solution in methanol (MeOH). Trypan blue solutions (4%) were purchased from Sigma-Aldrich (St. Louis, Mo.). RPMI 1640 medium solution, trypsin-ethylenediaminetetraacetic acid (Trypsin-EDTA) (0.025%), penicillin-streptomycin and fetal bovine serum (FBS) were obtained from Life Technologies (Grand Island, N.Y.). lonomycin (Calcium salt, Sigma Chemical Co.) was used to saturate the Ca²⁺-Fluo-4 fluorescence within the cells. Ionomycin was dissolved in DMSO to make the stock solution which was finally diluted in HBSS containing 1 mM CaCl₂ to make working solutions. The glioma cells (U-87 MG) were obtained from ATCC (Manassas, Va.). The cells were maintained in the RPMI medium with 10% fetal bovine serum and 1% penicillin in a 5% CO₂ atmosphere at 37° C. and were passaged twice a week.

Instrument

An optical imaging and fluorescent measurement system was used, as previously described (Li et al., 2009). Briefly, an inverted microscope (TE300, Nikon, Mississauga, ON, Canada) was connected to a video camera (JVC, TK-C3180). A TV set was used for optical observation (FIG. 2). By using a dichroic filter (620 nm), only red light entered the video camera for cell imaging without interfering with the fluorescent measurement. The green fluorescent emission (535 nm) was achieved under the Xenon arc lamp excitation (480 nm) selected by the microphotometer system (PTI) through a detection aperture. The chip was translated back and forth manually so the cellular fluorescence or background signal could be measured.

Isolation of a Single Glioma Cell

Before running any experiment, the microfluidic chip was cleaned by soap solution (2 times), rinsed with deionized water (3 to 5 times), and sterilized with 75% ethanol (1 time). After the cleaning step, 54 of a cell suspension was introduced into the cell inlet from reservoir 1, the cells flowed from the left to right across the cell retention structure. By adjusting the liquid levels of the right reservoir 2 (waste) and the left reservoir 1 (cell inlet), a desired U-87 MG cell was slowed down near the entrance of the cell retention structure. As the glioma cell is adherent, it readily becomes stationary to maintain its location in the retention structure. The glioma cell was allowed to settle for about 15 minutes, during which it was attached to the glass chip surface before the fluorescence measurement started. Before running the experiment, all the medium was removed and new medium was introduced from reservoir 1 to make sure the target glioma cell did not move during the experiment.

On-Chip Dye Loading

As soon as the cell was attached to the glass chip surface, the cell medium in all reservoirs was removed and Fluo-4 AM (5 μM) solution was introduced from the middle reservoir 2 for on-chip dye loading. Meanwhile, fluorescence measurement was used to monitor the on-chip dye loading process. The Fluo-4 AM dissociated after hydrolysis by the cellular esterase to give Fluo-4; the fluorescence was caused by binding of Fluo-4 to the basal level of Ca²⁺ ions. According to the fluorescence intensity, 10-12 min (or 600-700 s) were enough to complete the hydrolysis of the Fluo-4 AM ester inside the cells. This on-chip dye loading method has been proven to minimize the cell damage that would result from the use of a centrifuge in the conventional off-chip dye loading procedure (Huang et al., 2015). As the fluorescence intensity is related to the calcium concentration, the free cytosolic calcium concentration is calculated from by the following equation (Takahashi et al., 1999).

$$[ {Ca}^{2 +} ] = {K}_{d} ( \frac{F - {F}_{m i n}}{{F}_{m a x} - F} ) \qquad ( 1 )$$

where F is the total fluorescence when the cell is in the aperature, Fₘᵢₙ is the background fluorescence when the cell is out of the aperature, and Fₘₐₓ is the cellular fluorescence maximum obtained by ionomycin. K_{d} is the dissociation constant of the dye (for Fluo-4, K_{d}=0.35 μM) (Gee et al., 2000).

On-Chip Intracellular Calcium Fluorescence Measurement

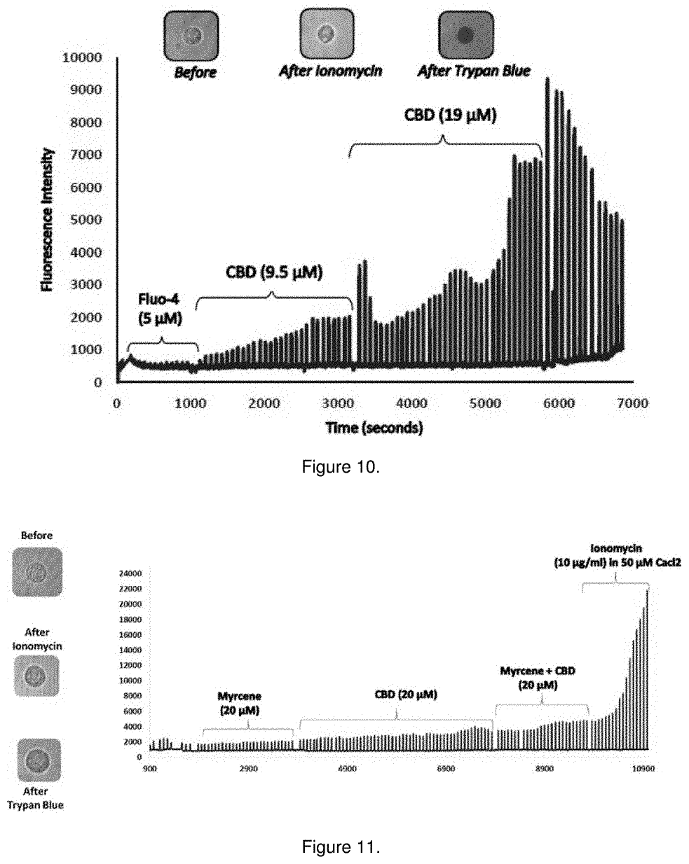

After Fluo-4 loading, the intracellular calcium ion concentration was measured at room temperature in the dark. Different concentrations of CBD (9.5 and 19 μM) were used for cell treatments. During data collection, the chip was translated back and forth so that the detection window monitored the cell and its surrounding region (the background) in turn. When the cell was inside the detection window, the cellular signals were recorded; whereas when the cell was outside the window, the signals from the background were obtained (FIG. 10). At the end of each experiment, a 10 μM of ionomycin solution was introduced to saturate calcium inside the cell to reach the maximum cellular fluorescence (Fₘₐₓ).

Cellular Response to Cannabinoids

This study was conducted to measure intracellular calcium concentrations ([Ca²⁺]ᵢ) induced by Cannabis-derived compounds in glioma cells. In order to monitor the fluorescence signals of a single glioma cell for Ca²⁺, the cell was treated by 5 μM of Fluo-4 AM which turned into Fluo-4 in the cell. Thereafter, the cell was treated with two different concentrations of CBD. Results demonstrated that exposure of CBD to the U-87 MG cell significantly augmented the intracellular [Ca²⁺]ᵢ levels in a concentration-dependent manner as shown in FIG. 10, i.e. the higher is the concentration, the stronger is the fluorescence signal intensity. Ionomycin saturated the Fluo-4 dye with calcium ions, and so immediately after adding ionomycin to the cell an obvious fluorescence peak was observed at ˜6000 s. According to FIG. 10, before adding CBD, [Ca²⁺]ᵢ was almost negligible, but after 9.5 μM CBD was added, [Ca²⁺]ᵢ increased slightly. Moreover, 19 μM CBD significantly increased [Ca²⁺]ᵢ in glioma cell compared to 9.5 μM. It seems that CBD at 19 μM generated the highest level of intracellular concentration from glioma cell. These observations are consistent with other reports that CBD showed antiproliferative effects on U87 and U373 human glioma cell lines, and that CBD exposure to cells reduced the mitochondrial oxidative metabolism and prohibited the viability of U87 human glioma cells in mice (Massi et al., 2004). In addition, CBD inhibited the translocation of U87 human glioma cells in vitro dose-dependently (0.01 up to 9 μM) (Massi et al., 2004; Vaccani et al., 2005).

In order to monitor [Ca²⁺]ᵢ dynamics in real time, the fluorescence intensity was converted to [Ca²⁺]ᵢ using eq. 1, using the calcium-free background fluorescence as Fₘᵢₙ, and ionomycin-saturated cellular fluorescence as Fₘₐₓ. According to fluorescence intensity obtained from FIG. 10, after 9.5 μM CBD was added, [Ca²⁺]ᵢ was increased to 47.9 nM, which was followed by the second noticeable increase, up to 913 nM, after treating the cell with 19 μM CBD. Finally, the target cell was found to be not completely stained after adding trypan blue which showed the cell was not dead by the end of the experiment.

Cellular Response to Cannabinoid in Combination with Terpene

As shown in FIG. 11, myrcene (20 μM) alone induced a little cell calcium response in the U-87 MG cell. The response from 10 μM myrcene was not detectable (data not shown). However, a combination of myrcene and CBD produced a higher cellular response as compared to CBD alone or myrcene alone (FIG. 11). The result of this single-cell experiment is consistent with the entourage effect of cannabinoid and terpene reported previous (Pellati et al., 2018).

Various embodiments disclosed herein are directed to a microfluidic single-cell method for monitoring the response of a single cell upon treatment of CBD and myrcene. The monitoring was based on the real-time measurement of intracellular calcium. Results indicated that CBD and myrcene significantly increased the [Ca²⁺]ᵢ levels in a dose-dependent fashion, based on calculations of the intracellular calcium concentration from a glioma cell within a microfluidic method.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to the present invention. Any priority document(s) and all publications, including but not limited to patents and patent applications, cited in this specification are incorporated herein by reference as if each individual publication were specifically and individually indicated to be incorporated by reference herein and as though fully set forth herein. The invention includes all embodiments and variations substantially as hereinbefore described and with reference to the examples and drawings.

### REFERENCES

Lewis M M, Yang Y, Wasilewski E, Clarke H A, Kotra L P. Chemical Profiling of Medical Cannabis Extracts. ACS omega. 2017; 2(9):6091-103.
Baron E P. Medicinal Properties of Cannabinoids, Terpenes, and Flavonoids in Cannabis, and Benefits in Migraine, Headache, and Pain: An Update on Current Evidence and Cannabis Science. Headache. 2018; 58(7):1139-86.
Duke J A, Duke J A. Handbook of medicinal herbs. CRC Press; 2002.
Mechoulam R, Lander N, Dikstein S, Carlini E A, Blumenthal M. On the Therapeutic Possibilities of Some Cannabinoids. The Therapeutic Potential Of Marihuana. Boston, Mass.: Springer US; 1976. pp. 35-45.
Pryce G, Baker D. Emerging properties of cannabinoid medicines in management of multiple sclerosis. Trends Neurosci. 2005; 28: 272-6.
Fichna J, Bawa M, Thakur G A, Tichkule R, Makriyannis A, McCafferty D-M, et al. PLoS One. Public Library of Science; 2014; 9: e109115.
Costa B, Colleoni M, Conti S, Parolaro D, Franke C, Trovato A E, et al. Oral anti-inflammatory activity of cannabidiol, a non-psychoactive constituent of cannabis, in acute carrageenan-induced inflammation in the rat paw. Naunyn-Schmiedebergs Arch. Pharmacol. 2004; 369: 294-9.
Devinsky O, Marsh E, Friedman D, Thiele E, Laux L, Sullivan J, et al. Lancet Neurol. 2016; 15: 270-8.
Stampanoni Bassi M, Sancesario A, Morace R, Centonze D, Iezzi E. Cannabinoids in Parkinson's Disease. Cannabis cannabinoid Res. Mary Ann Liebert, Inc.; 2017; 2: 21-29.
Wang M, Wang Y H, Avula B, Radwan M M, Wanas A S, Mehmedic Z, et al. Journal of forensic sciences. 2017; 62(3):602-11.
Mahmoud El Sohly A (2014) Constituents of Cannabis Sativa. Handbook of Cannabis.
Appendino, G., Chianese, G., &Taglialatela-Scafati, O. (2011). Current Medicinal Chemistry, 18(7), 1085-1099.
Ross, S. A., & Elsohly, M. A. (1996). Journal of Natural Products, 59(1), 49-51.
Vanhoenacker, G., Van Rompaey, P., De Keukeleire, D., & Sandra, P. (2002). Natural Product Letters, 16(1), 57-63.
Turner, C. E., & Elsohly, M. A. (1976). Lloydia, 39(6), 474-1474.
Brenneisen, R. (2007). Chemistry and analysis of phytocannabinoids and other Cannabis constituents. In M. A. Elsohly (Ed.), Marijuana and the cannabinoids (pp. 17-49). Totowa: Humana Press.
Russo E B. Taming THC: potential Cannabis synergy and phytocannabinoid-terpenoid entourage effects. British Journal of Pharmacology. 2011; 163(7):1344-64.
Richins R D, Rodriguez-Uribe L, Lowe K, Ferral R, O'Connell M A. Accumulation of bioactive metabolites in cultivated medical Cannabis. PloS one. 2018; 13(7):e0201119.
Leghissa A, Hildenbrand Z L, Foss F W, Schug K A. Journal of separation science. 2018; 41(2):459-68.
Mariotti Kde C, Marcelo M C, Ortiz R S, Borille B T, Dos Reis M, Fett M S, et al. Science & Justice: Journal of the Forensic Science Society. 2016; 56(1):35-41.
Cadola L, Broseus J, Esseiva P. Forensic science international. 2013; 232(1-3):e24-7.
Hillig K W, Mahlberg P G. American journal of botany. 2004; 91(6):966-75.
Ilias, Y., Rudaz, S., Mathieu, P., Veuthey, J., & Christen, P. (2004). Chimia, 58, 219-221.
Bruci Z, Papoutsis I, Athanaselis S, Nikolaou P, Pazari E, Spiliopoulou C, et al. Forensic science international. 2012; 222(1-3):40-6.
Vanhove W, Van Damme P, Meert N. Forensic science international. 2011; 212(1-3):158-63.
Tipparat P, Natakankitkul S, Chamnivikaipong P, Chutiwat S. Forensic science international. 2012; 215(1-3):164-70.
Tipparat, Prapatsorn, SuthatJulsrigival, Sarita Pinmanee and SurapolNatakankitkul. “Classification of Cannabis Plants Grown in Northern Thailand using Physico-Chemical Properties.” (2014).
Phillips R, Turk R, Manno J, Jain N, Forney R. Seasonal variation in cannabinolic content of Indiana marihuana. Journal of forensic sciences. 1970; 15(2):191-200.
Booth, J. K.; Bohlmann, J. Terpenes in Cannabis sativa—from plant genome to humans. Plant Sci. 2019, 284, 67-72
Ibrahim, E., Wang, M., Radwan, M., Wanas, A., Majumdar, C., Avula, B., Wang, Y. H., Khan, I., Chandra, S., Lata, H., Hadad, G., Abdel Salam, R., Ibrahim, A., Ahmed, S., ElSohly, M. Planta Med. 2019, 85, 431-438.
King, J. W. Curr. Opin. Food Sci. 2019, 28, 32-40.
Aizpurua-Olaizola, O., Soydaner, U., Ozturk, E., Schibano, D., Simsir, Y., Navarro, P., Etxebarria, N., Usobiaga, A. J. Nat. Prod. 2016, 79, 324-331
Gallily, R., Yekhtin, Z., Hanuš, L. O., The Anti-inflammatory properties of terpenoids from cannabis. Cannabis Cannabinoid Res. 2018, 3, 282-290
Casano, S., Grassi, G., Martini, V., Michelozzi, M., Variations in terpene profiles of different strains of Cannabis sativa L. Acta Hortic. 2011, 925, 115-121.
Arnoldi, S.; Roda, G.; Casagni, E.; Dell'acqua, L.; Cas, M. D.; Fare, F.; Rusconi, C.; Visconti, G. L.; Gambaro, V., Characterization of the Volatile Components of Cannabis Preparations bySolid-Phase Microextraction Coupled to Headspace-Gas Chromatographywith Mass Detector (SPME-HSGC/MS). Journal of Chromatography & Separation Techniques 2017, 8, 1-6.
Booth, J. K.; Page, J. E.; Bohlmann, J., Terpene synthases from Cannabis sativa. PloS one 2017, 12 (3), e0173911.
Calvi, L., Pentimalli, D., Panseri, S., Giupponi, L., Gelmini, F., Beretta, G., Vitali, D., Bruno, M., Zilio, E., Pavlovic, R., Giorgi, A., Comprehensive quality evaluation of medical Cannabis sativa L. inflorescence and macerated oils based on HS-SPME coupled to GC-MS and LC-HRMS (q-exactive orbitrap®) approach. J. Pharm. Biomed. Anal. 2018, 150, 208-219
Pavlovic, R., Nenna, G., Calvi, L., Panseri, S., Borgonovo, G., Giupponi, L., Cannazza, G., Giorgi, A. Molecules 2018, 23, 1-22
Ronald Honnold, Robert Kubas, and Anthony Macherone. Analysis of Terpenes in Cannabis Using the Agilent 7697A/7890B/5977B Headspace GC-MSD System. 2017.
Duncan R S, Goad D L, Grillo M A, Kaja S, Payne A J, Koulen P. Molecules (Basel, Switzerland). 2010; 15(3):1168-95.
Gee K R, Brown K A, Chen W N, Bishop-Stewart J, Gray D, Johnson I. Cell calcium. 2000; 27(2):97-106.
Huang, H. C., P. Chang, S. Y. Lu, B. W. Zheng, and Z. F. Jiang. 2015. Journal of receptor and signal transduction research 35 (5):450-7.
Li X, Li P C. Analytical chemistry. 2005; 77(14):4315-22.
Li X, Xue X, Li P C H. Integrative Biology. 2009; 1(1):90-8.
Lu J, Ju Y T, Li C, Hua F Z, Xu G H, Hu Y H. Asian Pacific journal of tropical medicine. 2016; 9(3):288-92.
Massi P, Vaccani A, Ceruti S, Colombo A, Abbracchio M P, Parolaro D. The Journal of pharmacology and experimental therapeutics. 2004; 308(3):838-45.
Montana V, Sontheimer H. The Journal of neuroscience: the official journal of the Society for Neuroscience. 2011; 31(13):4858-67.
Pellati, F., et al. (2018). Molecules (Basel, Switzerland) 23(10): 2639.
Pisanti S, Malfitano A M, Ciaglia E, Lamberti A, Ranieri R, Cuomo G, et al. Pharmacology & therapeutics. 2017; 175:133-50.
Takahashi, A., P. Camacho, J. D. Lechleiter, and B. Herman. 1999. Physiological Reviews 79 (4):1089-125.
Vaccani A, Massi P, Colombo A, Rubino T, Parolaro D., Br J Pharmacol. 2005; 144(8): 1032-6.
Watkins S, Sontheimer H., Trends in neurosciences. 2012; 35(9):546-56.

1. A method of detecting more than one cannabinoid in a sample, the method comprising:
extracting the more than one cannabinoid from the sample using a first C₁-C₄ alcohol as an extraction solvent to produce a supernatant, drying the supernatant to produce a dried extract, and dissolving the dried extract in a second C₁-C₄ alcohol;
separating the more than one cannabinoid by gas chromatography using a capillary column with hydrogen as a carrier gas; and
detecting the more than one cannabinoid using a mass spectrometer.

2. The method of claim 1, wherein a flow rate of the carrier gas is constant at about 1.6 mL/minute.

3. The method of claim 1, wherein a temperature program of the column is an initial temperature of 180° C. for 0.5 minutes, a first ramp of 5° C./minute to 250° C., and a second ramp of 10° C./minute to a final temperature of 325° C. for 2 minutes.

4. The method of claim 1, further comprising quantifying the amount of CBD and/or THC in the sample using CBD-d3 and/or THC-d3, respectively, as internal standards.

5. The method of claim 1, further comprising quantifying the amount of CBC, CBG and/or CBN in the sample using a standard addition method.

6. (canceled)

7. The method of claim 1, wherein the first and second C₁-C₄ alcohols are methanol.

8. (canceled)

9. (canceled)

10. The method of claim 1, wherein detector port temperatures of the mass spectrometer are about 280° C. at a transfer line, about 230° C. at an ion source, and about 150° C. at a quadrupole.

11. The method of claim 1, wherein the sample contains five or more cannabinoids and five of the cannabinoids are identified in the sample.

12. The method of claim 1, wherein the extraction step comprises suspending the sample in the C₁-C₄ alcohol, vortexing, sonicating and centrifuging the sample to produce the supernatant and filtering the supernatant.

13. A method of detecting more than one terpene in a sample, the method comprising:
extracting the more than one terpene from the sample using a first C₅-C₈ solvent as an extraction solvent to produce a supernatant, drying the supernatant to produce a dried extract, and dissolving the dried extract in a second C₅-C₈ solvent;
separating the more than one terpene by gas chromatography using a capillary column with hydrogen as a carrier gas; and
detecting the more than one terpene using a mass spectrometer.

14. The method of claim 13, wherein a flow rate of the carrier gas is constant at about 1.6 mL/minute.

15. The method of claim 13, wherein a temperature program of the column is an initial temperature of 70° C., a first ramp of 10° C./minute to 90° C., a second ramp of 40° C./minute to 150° C., and a third ramp of 120° C./minute to a final temperature of 300° C.

16. (canceled)

17. The method of claim 13, wherein the first and second C₅-C₈ solvents are hexane.

18. (canceled)

19. The method of claim 13, wherein detector port temperatures of the mass spectrometer are about 280° C. at a transfer line, about 230° C. at an ion source, and about 150° C. at a quadrupole.

20. (canceled)

21. The method of claim 13, wherein the sample contains seven or more terpenes and seven of the terpenes are identified in the sample.

22.-25. (canceled)

26. A method of determining an effect of one or more Cannabis-derived compounds on intracellular calcium concentration in a cell, the method comprising:
isolating a cell in a microfluidic device;
measuring fluorescence of the cell to determine a background fluorescence (Fₘᵢₙ);
adding a cell-permeable fluorescent calcium indicator to a reservoir in the microfluidic device;
measuring fluorescence of the cell and determining a first intracellular calcium concentration in the cell according to equation (1):
$$[ {Ca}^{2 +} ] = {K}_{d} ( \frac{F - {F}_{m i n}}{{F}_{m a x} - F} ) \qquad ( 1 )$$
adding the one or more Cannabis-derived compounds to the reservoir in the microfluidic device;
measuring fluorescence of the cell and determining a second intracellular calcium concentration in the cell according to equation (1);
adding ionomycin to the cell;
measuring fluorescence of the cell to determine a maximum fluorescence (Fₘₐₓ); and
comparing the first intracellular calcium concentration to the second intracellular calcium concentration to determine the effect of the one or more Cannabis-derived compounds on intracellular calcium concentration in the cell.

27. The method of claim 26, wherein the Cannabis-derived compounds are cannabinoids and/or terpenes.

28. The method of claim 26, wherein the Cannabis-derived compound is CBD or CBD and myrcene.

29. (canceled)

30. The method of claim 26, wherein the cell-permeable fluorescent calcium indicator is Fluo-4 acetoxymethyl ester (Fluo-4 AM).

31. The method of claim 26, wherein the cell is a glioma cell.
